(19) United States
(12) Patent Application Publication
(10) Pub. No.: US 2004/0241117 A1
(43) Pub. Date: Dec. 2, 2004

(54) [Method, Substance and Kit For Applying Makeup]

(76) Inventor: Jodie Lynn Urchyshyn, Calgary (CA)

Correspondence Address: JOHN J. ELNITSKI, JR., 225 A SNOWBIRD LANE, BELLEFONTE, PA, 16823, US

(21) Appl. No.: 10/250,074

(22) Filed: Jun. 2, 2003

(51) Int. Cl. A61K 7/021

(52) U.S. Cl. 424/63

(57) ABSTRACT

A facial and body makeup substance includes a clear makeup liquid and a sparkles containing material, contained in a kit and applied to form at least a line with sparkles.

### BACKGROUND OF INVENTION

The present invention relates to a method for applying a facial and body makeup, as well as a substance for applying the makeup as well as a kit for applying the makeup.

Methods, substances and kits for applying facial and body makeups, for example to an upper eyelid and other parts of the body are well known in the art. Some of them are disclosed for example in U.S. Pat. Nos. 4,423,031; 5,013,543; 5,137,040; 5,688,493; 5,876,704; 6,139,827; 6,303,105 and 6,306,916. It is believed that the existing methods, substances and kits can be further improved.

Accordingly, it is an object of the present invention to provide a method, a substance and a kit for applying makeup which are further improvements of the existing methods, substances and kits.

In keeping with these objects and with others which will become apparent hereinafter, one feature of the present invention resides, briefly stated, in a method of applying a makeup which includes the steps of applying a clear makeup liquid; and applying a sparkles imitating material so as to create at least a line with sparkles.

It is another feature of the present invention to provide a substance for applying a makeup which includes a clear makeup liquid; and a sparkles imitating material in the clear makeup liquid to be applied for creating at least a line with sparkles.

Finally, it is another object of the present invention to provide a kit for applying a makeup which includes means containing a clear makeup liquid; and means containing a sparkles imitating material, so that the clear makeup liquid with the sparkles imitating material can be applied to create at least a line with sparkles.

The novel features which are considered as characteristic for the present invention are set forth in particular in the appended claims. The invention itself, however, both as to its construction and its method of operation, together with additional objects and advantages thereof, will be best understood from the following description of specific embodiments when read in connection with the accompanying drawings.

### SUMMARY OF INVENTION

A facial and body makeup substance includes a clear makeup liquid and a sparkles containing material, contained in a kit and applied to form at least a line with sparkles.

### BRIEF DESCRIPTION OF DRAWINGS

FIG. 1 is a view illustrating an inventive kit for applying a makeup which is used for an inventive method of applying the makeup with an inventive makeup substance.

### DETAILED DESCRIPTION

In accordance with the present invention a substance for applying a makeup is proposed. The inventive substance for applying a makeup includes a clear makeup liquid; and a sparkles imitating material to create at least a line with sparkles.

The clear makeup liquid can be composed of water, SDA-40, alcohol, acrylates/octylacrylamide/copolymer, triethanolamine, propylene, glycol, imidazolidinyl urea, methylparaben, propylparaben. The clear liquid makeup may further contain cellulose, polysorbate 80, diazolioinyl or cellulose gum. The clear makeup liquid composed of the above mentioned components is known under the trade name “Mehron” produced by Mehron, Inc. Of Chestnut Ridge, N.Y.

The substance further includes a sparkles imitating material. It can be composed for example of an acrylic polymer.

A user dips an applicator, such as for example a fine brush, into the clear makeup liquid, then takes the liquid laden brush, and dips it into a sparkles imitating material. By painting a line, the user can create at least a color line with sparkles.

The proposed makeup substance and the method of its application are especially favorable for applying a color to an upper eyelid, for example. With the inventive substance and method, sparkles can be easily applied by the user to the upper eyelid with a dramatic effect.

With the substance and method in accordance with the present invention, the sparkles adhere well and are easy to apply in fine line. The substance can be easily removed with water. It is also possible to provide the sparkle imitating material within the bottle of clear makeup liquid.

In accordance with a further feature of the present invention, a colored eyeliner makeup can be first applied to the area in which the sparkles are to be applied. Then, the clear makeup liquid with the sparkles imitating material is applied on top of the eyeliner makeup. This may enhance the desired effect and also provides an additional barrier of protection from the eye, for example.

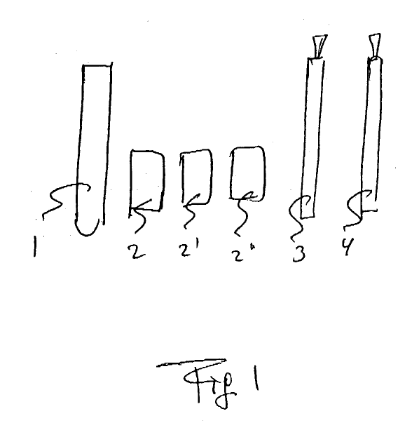

In order to apply the makeup substance in accordance with the inventive method, a kit can be provided as shown in FIG. 1. The kit contains a small container 1 with the clear makeup liquid specified herein above, at least one container with the sparkles imitating identified with reference numeral 2, and an applicator such as a fine brush identified with reference numeral 3. The kit is used as explained in detail herein above, in the description of the inventive method. In accordance with another embodiment of the present invention, it is possible to have several containers 2, 2′, 2″ etc with different sparkles imitating material. The different sparkles imitating material can differ in their color, design, etc.

In accordance with still a further feature of the present invention an additional applicator 4 can be provided for applying colored eyeliner cosmetic. In this case an eyeliner cosmetic container can be provided as well.

It will be understood that each of the elements described above, or two or more together, may also find a useful application in other types of methods and constructions differing from the types described above.

While the invention has been illustrated and described as embodied in method, substance and kit for applying makeup, it is not intended to be limited to the details shown, since various modifications and structural changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed as new and desired to be protected by Letters Patent is set forth in the appended claims:

1. A method of applying a makeup, comprising the steps of applying a clear makeup liquid; and applying a sparkles imitating material so as to create at least a line with sparkles.

2. A method as defined in claim 1, wherein said applying the clear makeup liquid includes applying the clear makeup liquid which is composed of water, SDA-40, alcohol, acrylates/octylacrylamide/copolymer, triethanolamine, propylene, glycol, imidazolidinyl urea, methylparaben, propylparaben.

3. A method as defined in claim 1, wherein said applying includes first dipping an applicator into the clear makeup liquid, introducing the applicator with the clear makeup liquid on it into the sparkles imitating material, and then applying by the applicator the clear makeup liquid together with the sparkles imitating material.

4. A method as defined in claim 1; and further comprising applying a colored makeup, and thereafter applying the clear makeup liquid and the sparkles imitating material.

5. A makeup substance, comprising a clear makeup liquid; and a sparkles imitating material introduced into the clear makeup liquid to be applied for creating at least a line with sparkles.

6. A makeup substance as defined in claim 5, wherein said clear makeup liquid is composed of water, SDA-40, alcohol, acrylates/octylacrylamide/copolymer, triethanolamine, propylene, glycol, imidazolidinyl urea, methylparaben, propylparaben.

7. A makeup substance as defined in claim 5, wherein the sparkles containing material includes an acrylic polymer.

8. A kit for applying a makeup, comprising means containing a clear makeup liquid; and means containing a sparkles imitating material, so that the clear makeup liquid with the sparkles imitating material can be applied to create at least a line with the sparkles.

9. A kit as defined in claim 8, wherein said means containing a clear makeup liquid is formed as a container containing the clear makeup liquid composed of water, SDA-40, alcohol, acrylates/octylacrylamide/copolymer, triethanolamine, propylene, glycol, imidazolidinyl urea, methylparaben, propylparaben.

10. A kit as defined in claim 8, wherein said means containing the sparkles imitating material include a container with the sparkles imitating material.

11. A kit as defined in claim 8, wherein said means containing the sparkles imitating material include a plurality of containers provided with different sparkles imitating materials.

12. A kit as defined in claim 8; and further comprising an applicator for taking the clear makeup liquid from said means containing the clear makeup liquid, introducing it into the means containing the sparkles imitating material, and then applying the clear makeup liquid with the sparkles imitating material.

13. A kit as defined in claim 8; and further comprising means for applying a colored eyeliner makeup before applying the clear makeup liquid with the sparkles imitating material.
